(19) United States
(12) Patent Application Publication
(10) Pub. No.: US 2008/0172004 A1
(43) Pub. Date: Jul. 17, 2008

(54) LUER ACTIVATED DEVICE WITH STRETCHABLE VALVE ELEMENT

(76) Inventor: MICHAEL PLISHKA, Lake Villa, IL (US)

Correspondence Address: BAXTER HEALTHCARE CORPORATION, 1 BAXTER PARKWAY, DF2-2E, DEERFIELD, IL, 60015, US

(21) Appl. No.: 11/550,582

(22) Filed: Oct. 18, 2006

(51) Int. Cl. A61M 5/00

(52) U.S. Cl. 604/249

(57) ABSTRACT

The present invention generally discloses a luer activated devices (LAD) that allows for the bidirectional transfer of fluids to and from medical fluid flow systems. The LAD includes a valve member that is highly elastic and stretchable and can include one or more apertures that are normally sealed when the valve member is in a relaxed or unstretched state. Fluid flow through valve member can occur when valve member 30 is stretched by the insertion of the male luer which causes the aperture to open without passing therethrough.

### FIELD OF THE INVENTION

The present invention relates generally to luer activated devices or valves that allow for the bidirectional transfer of fluids to and from medical fluid flow systems.

### BACKGROUND OF THE INVENTION

Luer activated devices (LAD) or valves (LAV) are commonly used in association with medical fluid containers and medical fluid flow systems that are connected to patients or other subjects undergoing diagnostic, therapeutic or other medical procedures. A LAD can be attached to or part of a fluid container or a medical fluid flow system to simplify the addition of fluids to or withdrawal of fluids from the fluid flow system.

Within the medical field there are a wide variety of medical fluid flow systems, serving a variety of functions. One of the more common uses of LADs are in association with fluid flow systems that are used for the intravenous administration of fluids, such as saline, antibiotics, or any number of other medically-related fluids, to a patient. These flow systems are commonly referred to as intravenous or “IV” fluid administration sets, and use plastic tubing to connect a phlebotomized subject to one or more medical fluid sources, such as intravenous solution or medicament containers.

Typically, such intravenous administration sets include one or more LADs providing needless access to the fluid flow path to allow fluid to be added to or withdrawn from the IV tubing. The absence of a needle for injecting or withdrawing fluid has the important advantage of reducing the incidence of needle stick injuries to medical personnel. A LAD typically includes a tapered female luer component, such as the inlet into a valve housing, that accepts and mates with a tapered male luer of a medical infusion or aspiration device, such as a needleless syringe or an administration set tubing brand.

There are certain characteristics and qualities of LADs that are highly desirable. For example, the LAD should provide a sufficient microbial barrier for the full service life of the valve. It is desirable that the microbial barrier be conducive to the application of standard aseptic techniques preformed by clinicians during the use of the device. For example, the geometry of the LAD should be such that it is easily swabbable and reduces the potential of entrapping particulates or contaminants that cannot be cleanly swabbed clear prior to use.

Furthermore, it is highly desirable that the LAD be substantially devoid of any interstitial space or any other “dead space” that cannot be flushed, or that such interstitial space be physically isolated from the fluid flow path. Such interstitial space has the potential of providing an environment for undesired microbial growth. In addition, the LAD should have a geometry that allows it to be sufficiently flushed so as to clear the dynamic fluid path and adjacent areas of residual blood or intravenous fluids to prevent undesired clotting or microbial growth.

LAD's are commonly used with intravenous catheters that provide access to a patient's vascular system. In such systems, another desirable feature of a LAD is minimal displacement of fluid during insertion and removal of the male luer. In certain situations, it is preferable that the LAD be a neutral/neutral device in that there is zero or only a very slight displacement of fluid during both insertion and removal of the male luer. In other situations it can be desirable for the LAD to produce a positive displacement of fluid from the valve housing during the insertion and removal of the male luer. The LAD also preferably prevents blood reflux into the catheter. Reflux is known to reduce the efficiency of the catheter and contribute to catheter clotting.

In most situations it is preferred that the LAD be ergonomically dimensioned to be completely activated by a wide range of ISO compliant male luer lock adaptors. However, there may some instances when the LAD is specifically designed to be activated by a male luer lock that is not ISO complaint. Another desirable characteristic of a LAD is the ability of the LAD to seal against pressure contained within a fluid system to which the LAD is connected. For example, it is desirable to be leak resistance to positive pressures ranging from 10 to 45 psi and to negative pressures or vacuum from 1 to 5 psi. The LAD also preferably has a geometry that allows for easy priming and flushing that does not require any additional manipulations to remove residual air bubbles from the tubing system.

These and other desirable characteristics, which may be used separately or in combination, is preferably present over the full service life of the valve. When used in connection with an IV set or catheter, the LAD may go through many connections and disconnections. It is desirable that the life of an LAD last through upwards to about 100 connections and disconnections or 96 hours of dwell time.

As described more fully below, the fluid access devices of the present invention provides important advances in the safe and efficient administration or withdrawal of medical fluids to or from a fluid flow system.

### SUMMARY OF THE INVENTION

In accordance with one aspect of the present invention, a medical valve is provided with a valve housing having an inlet adapted for receiving a male luer, an outlet and a flow path therethrough. The medical valve also includes a stretchable membrane that seals the flow path of the valve housing. The stretchable membrane includes at least one resealable opening having a normally closed configuration preventing the flow of fluid through the medical valve and an open configuration that allows the flow of fluid through the medical valve. The resealable opening changes from the closed configuration to the open configuration upon stretching of the stretchable membrane. When a male luer is inserted into the opening of the valve inlet, the male luer stretches the stretchable membrane without passing therethrough, thereby changing the resealable opening from the normally closed position to the open position.

According to another aspect of the present invention, a medical valve is provided with a valve housing inlet adapted for receiving a male luer, an outlet and a flow path therethrough. The medical valve also includes a valve member associated with the inlet and having a stretchable membrane including at least one resealable aperture. The resealable aperture has a normally closed configuration for preventing fluid flow and an open configuration that allows fluid flow. The resealable aperture opens upon stretching of the stretchable membrane by a male luer inserted into the housing. The male luer stretches the stretchable membrane without passing therethrough.

According to yet another aspect of the present invention, a method of transferring fluid from a male luer to a medical valve. The method comprises inserting a male luer into an opening of an inlet of a medical valve that is sealed by a stretchable member including at least one resealable opening. The resealable opening is closed when the stretchable member is in a relaxed state and opened when the stretchable member is stretched. The method further includes stretching the stretchable member by insertion of a male luer to open the resealable opening without passing therethrough and transferring fluid through the male luer and medical valve.

### BRIEF DESCRIPTION OF THE DRAWINGS

In describing the preferred embodiments of the present invention, reference will be made to the accompanying drawings, wherein:

FIG. 1 is a cross-sectional view of one embodiment of a luer activated device employing the present invention and a male luer.

FIG. 2 is the luer activated device of FIG. 1 with a male luer inserted therein.

FIG. 3 is a cross-sectional view of the luer activated device of FIG. 1 taken at line 3-3 of FIG. 1 and showing one embodiment of a stretchable valve member.

FIG. 4 is a cross-sectional view of another embodiment of a luer activated device employing the present invention and a male luer.

FIG. 5 is a cross-sectional view of the luer activated device of FIG. 4 taken at line 5-5 of FIG. 4 and showing another embodiment of a stretchable valve member.

FIG. 6 is the luer activated device of FIG. 4 with a male luer inserted therein.

### DETAILED DESCRIPTION

It is to be understood that the present invention disclosed herein, may be embodied in various forms. Therefore, specific details described herein are not to be interpreted as limiting the present invention.

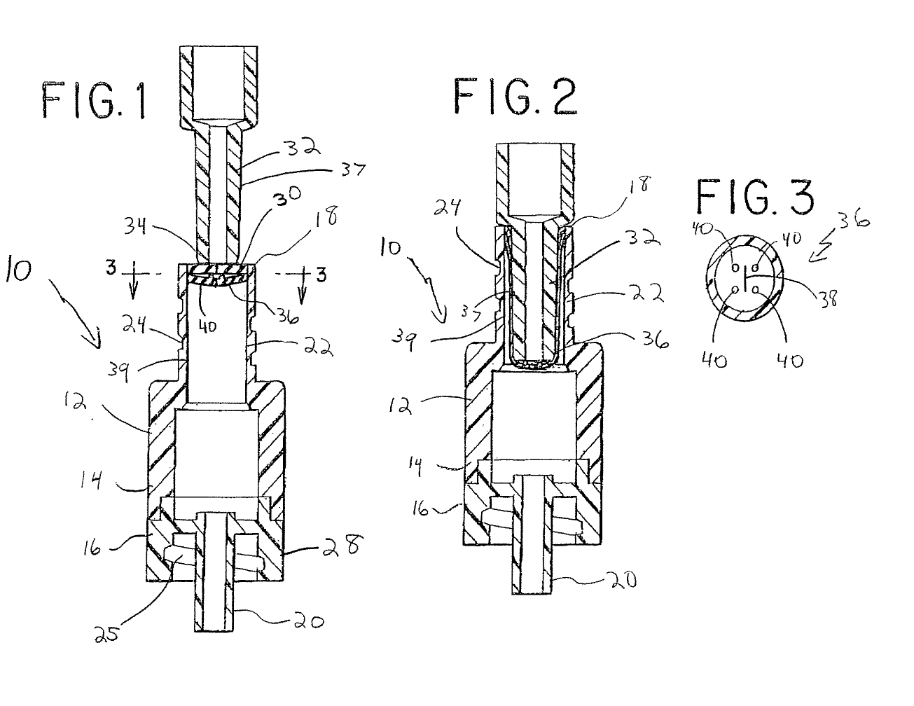

One embodiment of a luer activated device (LAD) of the present invention is shown generally in FIG. 1. LAD 10 includes a housing 12 that may include an upper portion 14 and a lower portion 16. Upper portion 14 can include an inlet 18 and bottom portion 16 can include an outlet 20. Typically, addition of fluids proceeds from inlet 18 downstream to outlet 20, but it should be noted that fluid may flow in other directions through the LAD, so “inlet” and “outlet” are used for relational or positional purposes only.

Inlet 18 can be made to conform with ISO and ANSI standards to receive a male luer also conforming with ISO and ANSI standards. In the embodiment shown in FIG. 1, inlet 18 includes neck 22 that can have threads 24 on an exterior surface for securing male luered device to the LAD 10. Alternatively, inlet 18 can also be specially made to receive certain other male luers or medical devices that do not conform to any particular standard.

Similarly, outlet 20 is preferably made to conform with international standards or can be specially designed to connect to any number fluid flow systems. Typically, outlet 20 can be connected to IV administrative tubing sets. To allow LAD 10 to be secured to an IV administration set, the bottom portion 16 can include threads 25 on an interior wall of skirt 28.

In accordance with the present invention, the LAD can include a valve to control fluid introduction or withdrawal therethrough. More particularly, as illustrated in FIG. 1, LAD 10 includes a first valve member 30 that is disposed to close inlet 18 and provide an antimicrobial barrier.

Valve member 30 is preferably made of latex or any suitable polymeric elastomer, such as silicone or similar material. The valve member 30 may be attached to the housing to seal inlet 18 in any suitable manner. For example, the valve member 30 may be bonded to the housing by adhesive or mechanically attached in any well known manner. The valve member 30 is preferably mounted to provide a smooth, flat surface at the inlet for ease of swabbing with a disinfecting agent, such as alcohol. For receiving a male luer, such as luer 32, the valve member 30 preferably has an aperture in the form of a slit 34, although the shape of the aperture is not critical.

Downstream or distal of the first valve member 30, the LAD 10 includes a second valve 36. As illustrated in FIG. 1, the second valve 36 is generally contiguous with the first valve member 30. The second valve member 36 is preferably made of highly stretchable elastic material defining a membrane that deforms upon contact with a male luer inserted into the inlet 18 and is suitable for contact with medical fluids. A preferred material for the second valve member 36 is a silicone elastomer having a durometer between 5 and 20 on the “A” scale and more preferably between about 7 and 15, still more preferably about 10 or thereabouts. The second valve member includes at least one normally closed aperture for fluid flow therethrough. As shown in FIG. 3, the aperture is in the form of a slit 38 that is normally closed when in the pre- or post-access position (FIG. 1) and opened by stretching of the membrane when the LAD 10 is accessed by male luer 32 for introduction or withdrawal of fluid. The second valve member 36 may also include one or more raised stand-off members in the form of protrusions 40 to prevent occlusions of the male luer as it presses against the valve member. The second valve member 36 may be attached housing 12 in any suitable manner. As shown in FIG. 1, the second valve member may be mounted to the housing immediately below the first valve member 30.

In use, upon insertion of a male luer 32 into the LAD 10, the luer first passes through slit 35 of the microbial barrier valve member 30 and immediately engages valve member 36. Further insertion of male luer 32 into the LAD causes the second valve member to extend and stretch, conforming generally around the male luer. This stretching also causes the slit 38 to open for introduction or withdrawal of fluid through the LAD 10. Because the second valve element 36 is stretched around and substantially covering the male luer 32, the external surface of the male luer 37 is substantially prevented from coming into contact with the internal fluid path through the valve housing or the internal wall 39 of the valve housing. This arrangement reduces the chances of cross-contamination between the male luer and the internal wall 39 of the housing.

The combination of the first and second valve members offers several benefits. The second valve member is resistant to significant back pressure when in the normally closed position (FIG. 1). This permits the first valve member to be made thinner than might otherwise be required and of higher durometer polymeric material, such as material having a durometer valve between about 30 and 90 and more preferably between 45 and 75 on the A scale.

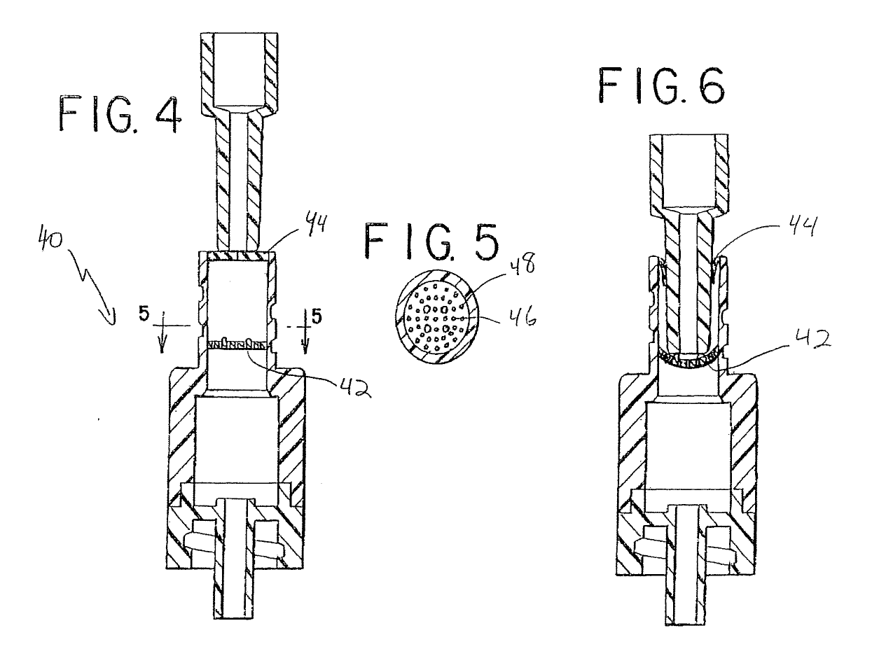

An alternative LAD 40 is shown in FIGS. 4-6. In many respects, LAD 40 is identical to that shown in FIGS. 1-3. However, in FIGS. 4-6, the second valve member 42 is spaced distally from the first valve member 44. As a result, less stretching of the second valve member 42 is needed when a male luer is inserted, as depicted in FIG. 6.

A further difference is evident in FIG. 5. The aperture in second valve member 42 in FIG. 5 is formed by a plurality of very small or micro apertures 46 that are normally closed and opened upon contact with and stretching of second valve member. As with the first embodiment, the second valve member 42 in FIG. 5 has a plurality of protrusions 48 to prevent occlusion of the male luer upon contact with the second valve member.

What is claimed is:

1. A medical valve, comprising:
a valve housing having an inlet, an outlet and a flow path therethrough, said inlet having an opening adapted for receiving a male luer;
a stretchable membrane sealing the flow path, said stretchable membrane including at least one resealable opening, said resealable opening having a normally closed configuration preventing the flow of fluid through the medical valve and an open configuration that allows the flow of fluid through the medical valve, said resealable opening changing from the closed configuration to the open configuration upon stretching of the stretchable membrane; and
wherein insertion of a male luer into the opening of the valve inlet stretches the stretchable membrane without passing therethrough, thereby changing the resealable opening from the normally closed position to the open position.

2. The medical valve of claim 1 further including a sealing member sealing the inlet of the valve housing, said sealing member disposed at a location proximal of the stretchable membrane, said sealing member having a resealable aperture for receiving a male luer therethough.

3. The medical valve of claim 2 in which the sealing member has a higher durometer value than the stretchable membrane.

4. The medical valve of claim 3 in which the stretchable membrane has a durometer of between about 5 A and about 20 A, and the sealing member has a durometer of about between 30 A and about 90 A.

5. The medical valve of claim 2 in which the sealing member and the stretchable membrane are contiguous.

6. The medical valve of claim 2 in which the stretchable membrane and the sealing member are spaced apart.

7. The medical valve of claim 6 in which the inlet of the valve housing includes a neck, said stretchable membrane being disposed within the neck and the sealing member being disposed at the opening.

8. The medical valve of claim 1 wherein the at least one resealable opening comprises a plurality of slits.

9. The medical valve of claim 1 wherein the at least one opening of the stretchable membrane comprises a plurality of openings.

10. The medical valve of claim 1 in which the at least one opening of the stretchable membrane comprises a plurality of micro openings.

11. The medical valve of claim 1 in which the stretchable membrane includes at least one standoff member for preventing occlusion of the male luer.

12. A medical valve, comprising:
a valve housing having an inlet, an outlet and a flow path therethrough, said inlet having an opening adapted for receiving a male luer; and
a valve member associated with the inlet and having a stretchable membrane, said stretchable membrane including at least one resealable aperture, said resealable aperture having a normally closed configuration preventing fluid flow and an open configuration that allows fluid flow, the at least one resealable aperture opening upon stretching of the stretchable membrane;
whereby insertion of the male luer stretches the stretchable membrane to open the at least one resealable aperture of the stretchable membrane without passing therethrough.

13. The medical valve of claim 12 further including a sealing member sealing the inlet of the valve housing, said sealing member disposed at a location proximal of the stretchable membrane, said sealing member having a resealable orifice for receiving a male luer therethough.

14. The medical valve of claim 13 wherein the sealing member has a higher durometer value than the stretchable membrane.

15. The medical valve of claim 13 wherein at least a portion of the sealing member and the stretchable membrane are contiguous.

16. The medical valve of claim 13 wherein the stretchable membrane and the sealing member are spaced apart.

17. The medical valve of claim 16 wherein the sealable membrane is positioned at the opening of the inlet and the stretchable membrane is positioned in the inlet and downstream from the sealing.

18. The medical valve of claim 12 wherein the at least one resealable aperture of the stretchable membrane comprises a plurality of openings.

19. The medical valve of claim 18 wherein the plurality of openings are a plurality of slits.

20. The medical valve of claim 12 wherein the at least one resealable opening of the stretchable membrane comprises a plurality of micro-openings.
